(19) United States
(12) Patent Application Publication
(10) Pub. No.: US 2003/0060792 A1
(43) Pub. Date: Mar. 27, 2003

(54) Light , absorbent pad with side leakage barrier system

(75) Inventors: John Harriz, Glen Mills, PA (US); Christa Bruce, Ardmore, PA (US); Ruth Levy, Collegeville, PA (US)

Correspondence Address: Caesar, Rivise, Bernstein,, Cohen & Pokotilow, Ltd., 1635 Market Street, 12th Floor, 7 Penn Center, Philadelphia, PA, 19120, US

(73) Assignee: Confab Services, AG

(21) Appl. No.: 09/957,906

(22) Filed: Sep. 21, 2001

(51) Int. Cl. A61F 13/15

(52) U.S. Cl. 604/385.04; 604/385.24; 604/378; 604/383

(57) ABSTRACT

A disposable absorbent article to trap and collect fluid waste products of a wearer. The pad includes a non-woven top sheet, a fluid acquisitions system, a core, and a polymer cover sheet. The top sheet includes a pair of elasticized flaps extending along its marginal sides, and which are secured in place at the respective ends of the pad, but which stand up on either side of the central portion of the pad so that the pad assumes a cup-like shape. The flaps prevent the egress of fluid laterally from the pad.

### FIELD OF THE INVENTION

This invention relates generally to disposable absorbent articles and more specifically to disposable absorbent articles, e.g., light incontinent pads, light menstrual cycle pads, and the like, that are flexible in the interest of comfort while exhibiting enhanced resistance to leakage at their longitudinal marginal sides.

### BACKGROUND OF THE INVENTION

Disposable absorbent sanitary articles, e.g., such as pantiliner pads, frequently make use of side flaps or “cuffs” to prevent lateral leakage of fluid from the pad. For example, U.S. Pat. No. 5,843,067 (Trombetta et al.) discloses an absorbent article including a containment assembly having a top sheet (e.g., woven and non-woven materials, polymeric materials such as apertured formed thermoplastic films, apertured plastic films, hydroformed thermoplastic films, porous foams, reticulated foams, reticulated thermoplastic film, thermoplastic scrims), a back sheet (e.g., a plastic film) and an absorbent core (e.g., comminuted wood pulp, creped cellulose wadding, meltblown polymers, chemically stiffened, modified or cross-linked cellulosic fibers, synthetic fibers, peat moss, tissue, absorbent foams, absorbent sponges, superabsorbent polymers and absorbent gelling materials). The back sheet is joined to the top sheet with the absorbent core positioned between the top sheet and the back sheet. The absorbent core has longitudinal edges and end edges. A containment cuff extends along each longitudinal edge of the absorbent core. The containment cuff includes a resilient member, an outer wrap (e.g.,woven and non-woven materials, apertured formed thermoplastic films, apertured plastic films, hydroformed thermoplastic films, reticulated thermoplastic film, thermoplastic scrims) covering the resilient member, and a stem portion for securing the containment cuff to the containment assembly. The outer-wrap may be either hydrophilic or hydrophobic depending on the material selected for use as the resilient member. If it is desired that the resilient member have some absorptive capacity, it is preferred that at least a portion of the outer-wrap be hydrophilic such that fluid may penetrate the outer-wrap and reach the resilient member contained therein. The resilient member has a height and a cross-sectional area. The resilient member is formed from a material that will recover at least about 80% of its original volume after it is compressed about 20% of its original volume. The stem portion has a first edge, a second edge, and a height as measured from its first edge to its second edge. The first edge of the stem portion is secured to the containment assembly inboard of the longitudinal edge of the absorbent core. The containment cuff has a height of the resilient member per height of the stem portion of about 0.04 to about 100 centimeters per centimeter.

Other U.S. Letters patents disclosing absorbent articles or pads with flaps or side cuffs are: U.S. Pat. No. 4,490,148 (Beckestrom), U.S. Pat. No. 4,496,360 (Joffe et al.), U.S. Pat. No. 4,695,278 (Lawson), U.S. Pat. No. 4,704,116 (Enloe), U.S. Pat. No. 4,795,451 (Buckley), U.S. Pat. No. 5,167,653 (Igaue et al.), U.S. Pat. No. 5,415,644 (Enloe), U.S. Pat. No. 5,575,785 (Gryskiewicz et al.), U.S. Pat. No. 5,599,338 (Enloe), U.S. Pat. No. 5,649,917 (Roberts et al), U.S. Pat. No. 5,681,303 (Mills et al.), U.S. Pat. No. 5,827,387 (Reynolds et al.), U.S. Pat. No. 6,142,985 (Feist), and U.S. Pat. No. 6,152,907 (Widlund et al.).

While all of the foregoing absorbent articles may be suitable for their intended purposes, they nevertheless leave something to be desired from the standpoint of simplicity of construction, ease of manufacture and cost.

### SUMMARY OF THE INVENTION

This invention relates to a disposable absorbent article, e.g., pantiliner or other absorbent pad, arranged to be worn by a wearer to trap and collect fluid waste products of the wearer.

The pad is an elongated generally planar member having a periphery including a pair of longitudinal extending sides interconnecting respective ones of a pair of ends, The pad comprises a top-sheet, a fluid absorbent core (e.g., a pair of non-woven layers with a superabsorbent material interposed between the layers) and an moister impervious outer cover sheet. The core has a pair of marginal side edges, a front end and a back end. The top sheet has a pair of side edges, a front end, a back end, and a central zone and comprises a non-woven material (e.g., a hydrophobic material whose central zone is hydrophilic) spaced from the side edges of said top sheet.

The top sheet is disposed over the core with the central zone being contiguous with the core and the marginal side edges of the top sheet extending beyond the marginal side edges of the core to form a pair of flaps extending along respective sides of the core. Each of the flaps has a marginal edge.

The cover sheet comprises a plastic film and is disposed under the core. The cover sheet has a top end, a bottom end, and a pair of marginal side edges secured to respective ones of the flaps.

Each of the flaps is folded along a respective first fold line adjacent the marginal side edges of the core to overlie portions of the core. A portion of each of the flaps contiguous with the front end of the pad is secured in place and a portion of each of the flaps contiguous with the rear end of said pad is secured in place. An intermediate portion of each of the flaps is located adjacent the central zone and is free to project upward with respect to the pad.

In accordance with one exemplary aspect of this invention, the marginal edges of the flaps are elasticized along at least a portion thereof to cause the pad to assume an arcuate (e.g., cup-shaped) configuration, with the intermediate portions of the flaps standing upward with respect to the pad.

In accordance with another exemplary aspect of this invention, a fluid acquisition system is provided located in the pad interposed between the top sheet and the core to facilitate transfer of fluid into the core.

### DESCRIPTION OF THE DRAWING

FIG. 1 is a top plan view, partially broken away, of one exemplary embodiment of a disposable absorbent pad of this invention;

FIG. 2 is an enlarged sectional view taken along line 2-2 of FIG. 1;

FIG. 3 is an enlarged sectional view taken along line 3-3 of FIG. 1;

FIG. 4 is a top plan view, partially broken away, of another exemplary embodiment of a disposable absorbent pad of this invention;

FIG. 5 is an enlarged sectional view taken along line 5-5 of FIG. 4;

FIG. 6 is an enlarged sectional view taken along line 6-6 of FIG. 4;

FIG. 7 is a top plan view, partially broken away, of still another exemplary embodiment of a disposable absorbent pad of this invention;

FIG. 8 is an enlarged sectional view taken along line 8-8 of FIG. 7;

FIG. 9 is an enlarged sectional view taken along line 9-9 of FIG. 7;

FIG. 10 is a top plan view, partially broken away, of yet another exemplary embodiment of a disposable absorbent pad of this invention;

FIG. 11 is an enlarged sectional view taken along line 11-11 of FIG. 10; and

FIG. 12 is an enlarged sectional view taken along line 12-12 of FIG. 10.

### DETAILED DESCRIPTION OF THE PREFERRED EMBODIMENT

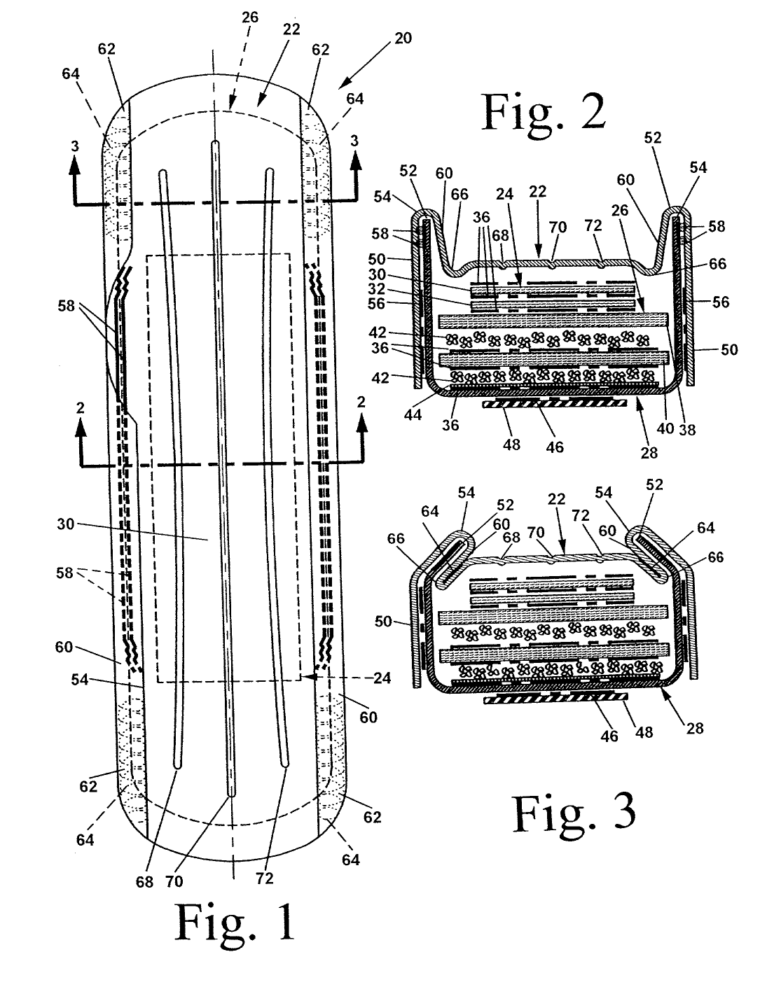

Referring now to the various figures of the drawing wherein like reference characters refer to like parts, there is shown in FIG. 1 a disposable absorbent article 20 constructed in accordance with one embodiment of this invention. It should be pointed out that as used herein the term “disposable” means that article is designed to be used until soiled, either by urination or otherwise, and then discarded, rather than being washed and used again.

In the embodiment of FIG. 1 and the other embodiments shown in the drawing, each article is in the form of a pantiliner pad. While the following description will focus on pantiliners, it should be clear that the subject invention can be used for any type of a padlike, absorbent article to be worn by a person within an undergarment for trapping urine or menses.

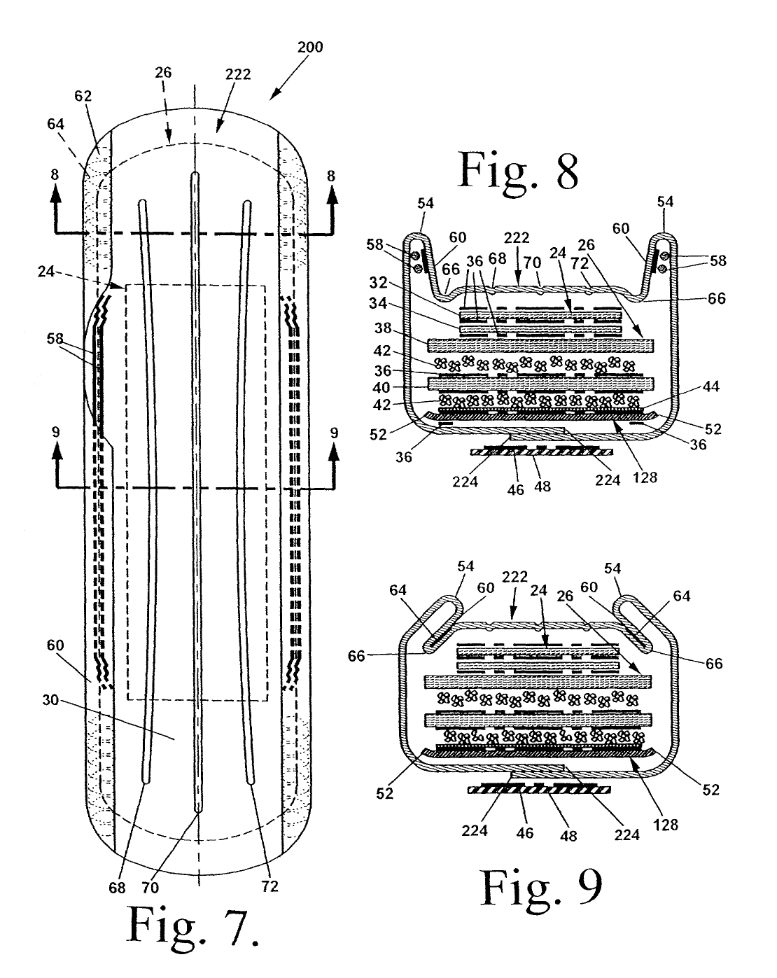

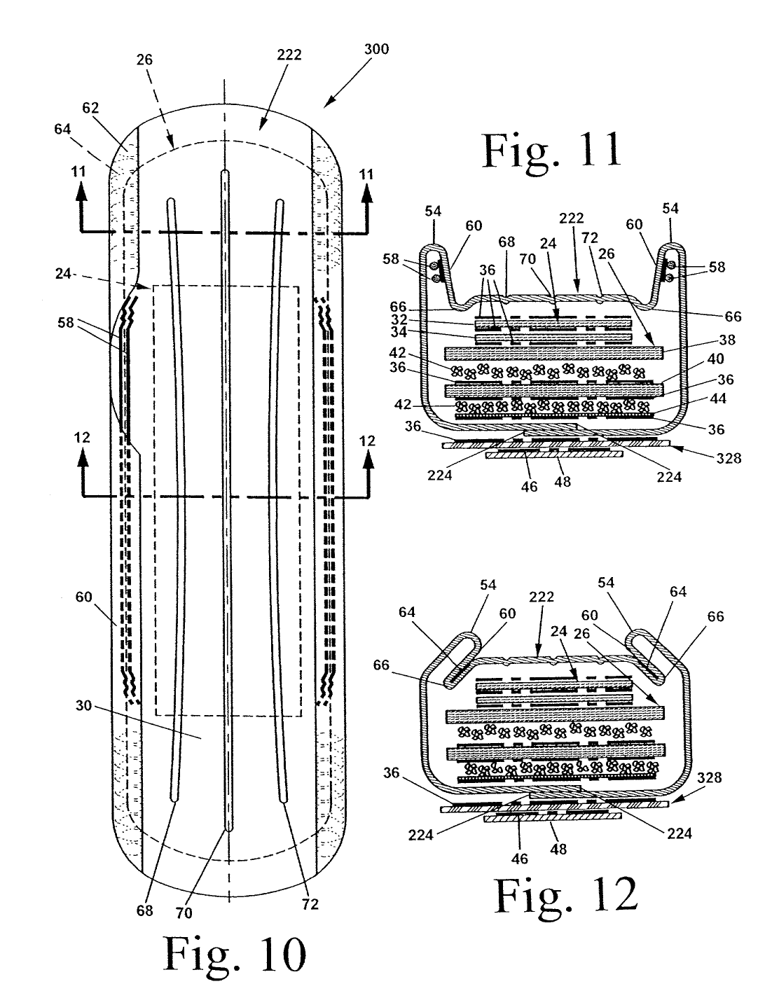

As can be seen in FIG. 1, the pantiliner 20 basically comprises a thin generally planar structure. In particular, the pad may be any thickness in the range of 2 mm to 12 mm, with 5.0 mm being one preferred thickness for a pantiliner. It should be pointed out at this juncture that the sectional views of FIGS. 2, 3, 5, 6, 8, 9, 11 and 12 are provided to illustrate the construction of the components and their arrangement and such views are not to scale (e.g., the pad is much thinner than shown). Moreover, these sectional views do not show the completed pad with its densified fluid resistant barrier lines that is shown in FIGS. 1, 4, 7 and 10 and which will be described later. The pad 20 is formed of a liquid pervious inner liner or top sheet 22, a fluid acquisition system 24, a liquid absorbent core 26, and an outer cover or moisture barrier 28. The top sheet 22 may be of any conventional material. In the embodiment 20 of FIG. 1 the top sheet 22 is formed of a hydrophobic non-woven material having a central portion or zone 30 which is treated to make it hydrophilic. The central portion is located at approximately the center of the pad and is of sufficient size to encompass the target or insult zone, i.e., the area that will receive the fluid insult from the pad's wearer. One particularly suitable material for the top sheet 22 is 15 gsm polypropylene spunbond, available from Avgol Ltd., Holon, Israel. The top sheet 22 may be formed of other materials than that described above, e.g., fibers of polyethylene, bi-component, polyester, rayon, cotton, etc., fiber combinations (e.g., spunbond, air laid, wet laid, carded, hydroentangled, etc.), and other basis weights may be used as well. In fact, if desired, the top sheet 22 may be formed of a liquid impermeable material, e.g., a three dimensional polymeric film, having plural apertures or pores extending therethrough so as to make the material liquid permeable. One particularly suitable polymeric film is that disclosed in U.S. Design Pat. No. 362,120, and co-pending U.S. application Ser. No. 09/439,793, filed on Nov. 12, 1999, entitled “Absorbent Article With Improved Fluid Acquisition System,” which patent and application are both assigned to the same assignee as this invention and whose disclosures are incorporated by reference herein. The top sheet of the other exemplary embodiments shown herein can be constructed in the same manner as the top sheet of the embodiment of FIG. 1.

A fluid acquisition system 24 is located under the top sheet 22 and over the absorbent core 26. The fluid acquisition system is arranged to manage, transport, accommodate and/or direct high volumes and flow rates of urine into the core 26 and can be of any type construction, e.g., one or more layers of a thru-air bonded/carded web, a spunbond bicomponent non-woven web, a web of crosslink cellulosic fibers, apertured 3D (three dimensional) film or the like. As best seen in FIGS. 2 and 3, one preferred example of the fluid acquisition system 24 basically comprises a pair of fluid transfer layers 32 and 34. The upper layer 32 is in the form of a three dimensional polymeric film having plural apertures or pores extending therethrough so as to make it liquid permeable, while the lower layer is preferably in the form of a non-woven material so that it is also liquid permeable. For example, the layers 32 and 34 can be constructed in accordance with the teachings in the aforementioned co-pending application Ser. No. 09/439,793. While the foregoing is a preferred construction for the fluid acquisition system 24, other fluid acquisition systems, e.g., conventional systems, can be used in the pad 20. In fact, if desired, the pad 20 need not include any fluid acquisition system.

The upper layer 32 of the fluid acquisition system 24 is secured by an adhesive 36 to the underside of the top sheet 22, while the lower layer 34 is secured to the upper layer by a similar adhesive 36. The adhesive 36 is preferably a low add-on adhesive. One particularly suitable adhesive is available from National Starch and Chemical of Bridgewater, N.J. under the trade designation Disposomelt 34-563A. The lower layer 34 is secured to the core 26 by another layer of adhesive 36.

The core 26 basically comprises a pair of absorbent material layers 38 and 40 with a granular layer of a super absorbent polymer powder (SAP) and a binder, the granular combination being designated by the reference number 42, interposed between the non-woven layers 38 and 40. Each of the absorbent layers can be made up of any conventional material used in conventional absorbent articles, such as pantiliners, as well as combinations of different types of absorbent material(s). For example, in the preferred embodiment shown in FIG. 1 both absorbent layers 38 and 40 of the absorbent core 26 are formed of an air-laid absorbent material, such as wood pulp. Examples of the SAP include polyacrylamides, polyvinyl alcohol, polyacrylates, various grafted starches, and the like. One particularly suitable super absorbent material is a cross-linked polysodium acrylate, which can be purchased from BASF Corporation, Portsmouth, Va., under the trade designation ASAP 2102. In the embodiment of FIG. 1 another granular layer 42 of the combination of SAP and binder is located under the lower layer 40 of the core 26. The layers of core 26 are adhesively secured together by interposed layers of adhesive 36 and the core as a whole is secured in place on the inner surface of the outer cover 28 by a low add-on adhesive 36 like that discussed above. If desired a tissue layer 44 may be located under the lower granular layer 42, such as shown in FIGS. 2 and 3.

In order to hold the pad 20 in place within the wearer's undergarment, it includes a plural stripes 46 (FIG. 3) of a “positioning” adhesive on the outer surface of the outer cover or moisture barrier 28. The stripes 46 extend along the longitudinal central axis of the pad 20 for substantially the length of the pad. Any suitable positioning adhesive can be used for the stripes, such as a pressure sensitive hot melt adhesive. One particularly suitable material for the positioning adhesive is available from National Starch and Chemical of Bridgewater, N.J. under the trade designation 34-5598. In order to protect the positioning adhesive stripes 46 from degradation or being soiled by debris, a single release strip 48 (e.g., a release paper) is releasably secured over the stripes. The release strip 48 can be formed of any suitable adhesive protective, yet easy to release, material. One particularly suitable material for the adhesive release strip 48 is silicon coated paper available from Loparex of Willobrook, Ill., under the trade designation ESP-39.

The pad 20 is arranged to naturally conform to the anatomy of the wearer. Moreover, the pad 20 includes flap portions (to be described hereinafter) which are elasticized to stand up somewhat from the plane of the otherwise flat pad along the marginal edges of the central zone to cause the pad to assume a cup-shaped configuration. To that end, as can be seen in FIGS. 2 and 3, the top sheet 22 is of a greater width than the outer cover 28 so that the marginal portions 50 of the top sheet 22 extend beyond the respective free edge 52 of the cover 28. Each marginal edge portion 50 is folded over an associated free edge of the cover at a first fold line 54 and is adhesively secured to the outer surface of the cover adjacent that first free edge 52 and fold line 54 via an adhesive 56. One particularly suitable adhesive 56 is available from National Starch under the designation Disposomelt 34-563A. A pair of pretensioned elastic threads 58 are located on the outer surface of the outer cover contiguous with one free edge 52 thereof, and an identical pair of elastic threads is located on the outer cover contiguous with the other free edge 52. The threads 58 are constructed of any suitable conventional material, such as Lycra, available from Dupont, Wilmington, Del., under the designation D 540. The threads extend along the central zone 30 of the pad 20 on opposite sides thereof and are pretensioned, e.g., prestretched, and adhesively secured in place by an adhesive. One particularly suitable adhesive is available from National Starch under the trade designation Disposomelt 34-564D.

As best seen in FIGS. 1 and 2, the portions of the top sheet 22 that overlie the inner surface of the cover 28 contiguous with the free edges 52 of the cover 28 effectively form a pair of flaps 60. Each flap 60 extends the length of the pad 20 on opposite sides of the pad. The end portions of the flaps, designated by reference number 62 in FIG. 1, i.e., the portions located at the opposite ends of the pad 20, are adhesively secured in place by an adhesive 64 as best seen in FIG. 3. This effectively forms a second (inner corner) fold line 68 (FIG. 3). Since the central portion of the flaps 60 are elasticized and pretensioned by the prestretched threads 58, when the pad is in its natural or relaxed state the flaps will effectively “stand up” from the plane of the pad at the fold line 66 like shown in FIG. 2 to cause the pad to assume an arcuate or cup-shaped configuration (as mentioned earlier). In particular, the pad will be bent so that its central portion or zone is concave in the longitudinal direction, with the elasticized flap portions extending upward from the pad on opposite sides of the concave portion contiguous with the fold lines 68. Since the end portions of the flaps are held down, this action effectively renders the pad ergonomically suited for disposition at the crotch of the wearer, with the top sheet 22 being located immediately adjacent the wearer's body.

Since the marginal portions 50 of the top sheet 22 extend over the marginal side portions of the outer cover 28 any portion of the wearer's body which the pad 20 contacts will be by the fibrous (non-woven) material of the pad and not the plastic film of the outer cover. This renders the pad non-sticky and very comfortable to wear. Moreover, the fact that the flaps are folded up and/or inward along the inner fold line 66 ensures that any fluid (e.g., urine and/or menses) introduced into the pad will not tend to migrate out of the pad from the sides of the pad at the insult or target zone.

In order to further deter the migration of fluid laterally out of the pad 20, the pad 20 preferably includes a plurality of generally parallel, fluid barrier lines 68, 70 and 72 (FIG. 1). Each of these barrier lines is arranged to form a somewhat dense wall across which fluid is deterred from flowing, while helping to direct or “channel” the fluid to flow therealong. Each of the lines 68, 70 and 72 is produced by applying pressure and/or heat to the portions of the pad 20 along the line to compress and increase the density of the materials along those lines, e.g., to compress the acquisition system layers and the core layers. Depending upon the construction of the pad, the compression and/or heat applied to the materials making up the pad (particularly its core and acquisition layer(s), if any) causes the interstitial spaces between the individual fibers making up the core (and any other layers of the pad composed of fibrous material) to compress or become densified to the point at which such densified areas are insufficient to allow liquid to flow therethrough. The densified lines 68, 70 and 72 through the layers of the pad are not shown in FIGS. 2 and 3 in the interest of drawing simplicity.

The application of pressure and heat to create the barrier lines 68, 70 and 72 can be accomplished using conventional thermal or ultrasonic bonding techniques or by pattern embossing. In some applications the use of pressure alone may be sufficient to produce a dense barrier line which remains after the pressure is removed. Moreover, an adhesive may be used when pressure is applied to create the dense barrier line. In fact, it is contemplated that water can be used in lieu of an adhesive for use with a core of suitable material so that after the pressure is released and the core dries the previously wet and compressed portions of the core will remain compressed, thereby forming the barrier lines.

As will be appreciated by those skilled in the art most leakage from a pad is along the marginal sides along the center portion of the pad. Thus, the provision of the flaps which stand-up along those portions of the pad is a great deterrent to the egress of liquid from the pad at those portions. The barrier lines provide further deterrence to liquid egress at those portions.

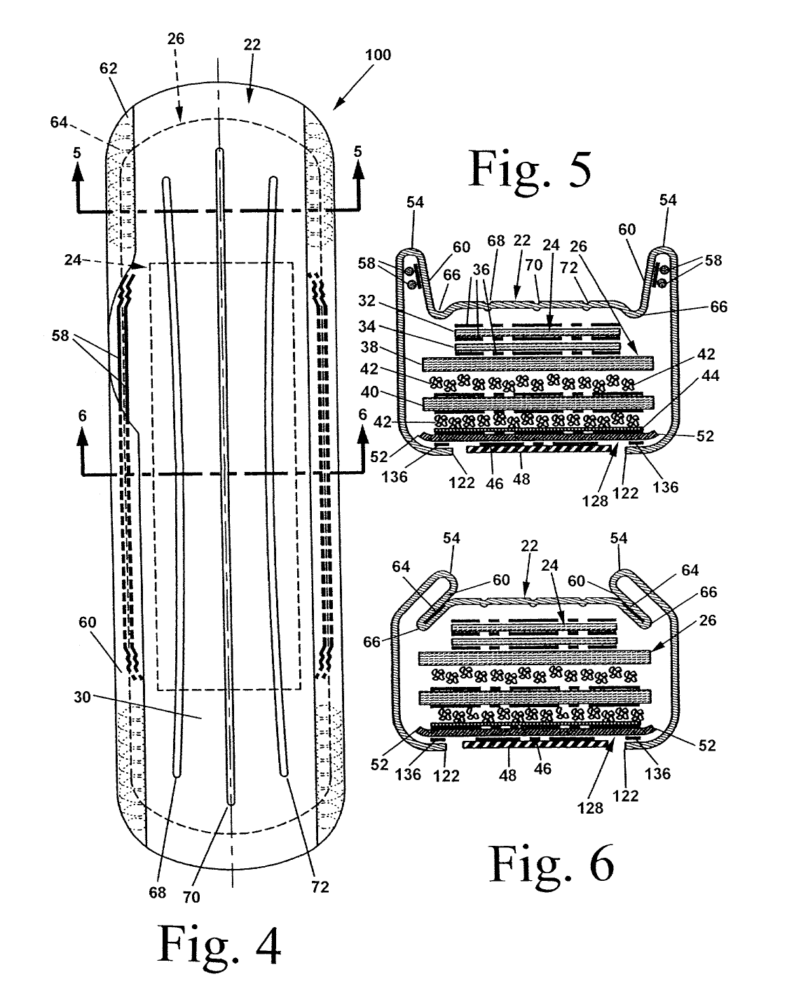

In FIG. 4 there is shown an alternative embodiment of a pantiliner pad 100 constructed in accordance with this invention. The pad 100 is identical in construction to the pad 20 described heretofore except for the fact that the outer cover 128 is only slightly wider than the width of the core 26. In the interest of brevity, the common components of pads 20 and 100 will be given the same reference numerals and their construction and arrangement will not be reiterated. As best seen in FIG. 5, no portion of the outer cover 128 extends into the flaps 60. In fact, the cover 128 is significantly narrower in width than the cover 28 described heretofore so that it only extends slightly beyond the sides of the core 26. The free ends 122 of the top sheet 22 are adhesively secured by their respective inner surfaces to the outer surface of the cover sheet 128 via a pair of longitudinally extending lines of adhesive 136. While this construction is somewhat less expensive and is cooler to wear than the pad 20, it provides less protection from lateral leakage since the flaps 60 do not include any portion of the moisture impervious outer cover therein. Thus, it is possible that some fluid may exit through the interstitial spaces of the fibrous material flaps under pressure notwithstanding that these portions of the non-woven are hydrophobic.

In FIG. 7 there is shown another alternative embodiment of a pantiliner pad 200 constructed in accordance with this invention. The pad 200 is identical in construction to the pad 100 except for the construction of the top sheet 222. Thus, in the interest of brevity, the common components of the pads 20, 100 and 200 will be given the same reference numerals and the details of their construction and arrangement will not be reiterated. In this embodiment the top sheet 222 of the pad 200 is wrapped entirely about the inner and outer surfaces of the pad and so that the top sheet's marginal edges 224 overlap. The cover 128 is not exposed (i.e., it is no longer “outer”).

As will be appreciated by those skilled in the art, the pad 200 provides even a higher level of wearer comfort than the foregoing pads since no portion of the pad has any exposed plastic film. However, the arrangement is more susceptible to fluid egress through the flaps 60 when under pressure since no portion of the plastic film of the cover 128 is located within the flaps. Of course, if the cover used is constructed like that of pad 20, i.e., a cover 28, egress of fluid through the pad's flap would be deterred by the presence of the plastic film therein.

In FIG. 10 there is shown still another alternative embodiment of a pantiliner pad 300 constructed in accordance with this invention. The pad 300 is identical in construction to the pad 200, except for the fact that the cover 328 (which is identical to cover 128 described above) is located (e.g., secured by adhesive 36) on the outside of the top sheet wrap 222 over the overlapping ends 224. As before, in the interest of brevity the common components of the pads 20, 100, 200 and 300 will be given the same reference numerals and the details of their construction and operation will not be reiterated.

As will be appreciated by those skilled in the art, the construction of pad 300 offers the advantage that the adhesive stripes 46 are easier to affix to a plastic film (as is the case with pad 300) than to a non-woven material (as is the case with pad 200).

In accordance with one exemplary aspect of this invention the longitudinal dimension of any of the pads, i.e., the maximum distance between the ends of any pad measured along the central longitudinal axis is approximately 11 inches (28 cm), whereas the lateral dimension measured transversely across the pad at the center of the pad is approximately 3.35 inches (8.5 cm).

It should be pointed out at this juncture that pads constructed in accordance with this invention can be of other sizes. Moreover, the pads can be of various shapes. Thus, the shape of the pads need not be the generally rounded rectangular shape as shown and described above, e.g., the pads may be of a “thong” shape, a “dog-bone” shape, etc.

Without further elaboration the foregoing will so fully illustrate our invention that others may, by applying current or future knowledge, adopt the same for use under various conditions of service.

We claim:

1. A disposable absorbent pad arranged to be worn by a wearer to trap and collect fluid waste products of the wearer, said pad being an elongated generally planar member having a periphery including a pair of longitudinal extending sides interconnecting respective ones of a pair of ends, said pad comprising a top-sheet, a fluid absorbent core, and an outer cover sheet, said core having a pair of marginal side edges, a front end and a back end, said top sheet comprising a non-woven material having a pair of side edges, a front end, a back end, and a central zone spaced from said side edges of said top sheet, said top sheet being disposed over said core with said central zone being contiguous with said core and said marginal side edges of said top sheet extending beyond said marginal side edges of said core to form a pair of flaps extending along respective sides of said core, each of said flaps having a marginal edge, said cover sheet comprising a plastic film and being disposed under said core, said cover sheet having a top end, a bottom end, and a pair of marginal side edges secured to respective ones of said flaps, each of said flaps being folded along a respective first fold line adjacent said marginal side edges of said core to overlie portions of said core, and wherein a portion of each of said flaps contiguous with said front end of said pad is secured in place and a portion of each of said flaps contiguous with said rear end of said pad is secured in place, and with an intermediate portion of each of said flaps being located adjacent said central zone and being free to project upward with respect to said pad.

2. The pad of claim 1 wherein said top sheet is formed of a hydrophobic material and wherein said central zone is hydrophilic.

3. The pad of claim 1 wherein said marginal edges of said flaps are elasticized along at least a portion thereof to cause said pad to assume an arcuate configuration, with said intermediate portions flaps standing upward with respect to said pad.

4. The pad of claim 2 wherein said marginal edges of said flaps are elasticized along at least a portion thereof to cause said pad to assume an arcuate configuration, with said intermediate portions flaps standing upward with respect to said pad.

5. The pad of claim 3 additionally comprising a fluid acquisition system interposed between said top sheet and said core.

6. The pad of claim 5 wherein said fluid acquisition system comprises an apertured film layer and a non-woven layer, with said apertured film layer being located contiguous with said top sheet and with said non-woven layer being located contiguous with said core.

7. The pad of claim 4 additionally comprising a fluid acquisition system interposed between said top sheet and said core.

8. The pad of claim 7 wherein said fluid acquisition system comprises an apertured film layer and a non-woven layer, with said apertured film layer being located contiguous with said top sheet and with said non-woven layer being located contiguous with said core.

9. The pad of claim 1 wherein each of said flaps is also folded along a second fold line spaced outward of said first fold line to form a double thickness flap, and wherein said second fold lines form said marginal edges of said flaps.

10. The pad of claim 9 wherein said flaps are elasticized by at least one elongated elastic threads, wherein each of said flaps is also folded along a second fold line spaced outward of said first fold line to form a double thickness flap, wherein said second fold lines form said marginal edges of said flaps, and wherein said at least one elastic thread is located in each of said double thickness flaps contiguous with said marginal edge thereof.

11. The pad of claim 2 wherein each of said flaps is also folded along a second fold line spaced outward of said first fold line to form a double thickness flap, and wherein said second fold lines form said marginal edges of said flaps.

12. The pad of claim 11 wherein said flaps are elasticized by at least one elongated elastic threads, wherein each of said flaps is also folded along a second fold line spaced outward of said first fold line to form a double thickness flap, wherein said second fold lines form said marginal edges of said flaps, and wherein said at least one elastic thread is located in each of said double thickness flaps contiguous with said marginal edge thereof.

13. The pad of claim 1 wherein said core comprises a pair of non-woven layer with a super absorbent material interposed therebetween.

14. The pad of claim 2 wherein said core comprises a pair of non-woven layer with a super absorbent material interposed therebetween.

15. The pad of claim 10 wherein respective ones of said marginal side edges of said cover sheet extend into respective ones of said flaps to a position adjacent said second fold of said flaps.

16. The pad of claim 12 wherein respective ones of said marginal side edges of said cover sheet extend into respective ones of said flaps to a position adjacent said second fold of said flaps.
